(19) United States
(12) Patent Application Publication
(10) Pub. No.: US 2003/0181880 A1
(43) Pub. Date: Sep. 25, 2003

(54) Self cleaning garment with portable cleaning apparatus suitable for incontinent individuals and the like

(76) Inventor: Steven Schwartz, Ithaca, NY (US)

Correspondence Address: BROWN & MICHAELS, PC, 400 M & T BANK BUILDING, 118 NORTH TIOGA ST, ITHACA, NY, 14850, US

(21) Appl. No.: 10/104,459

(22) Filed: Mar. 22, 2002

(51) Int. Cl. A61F 13/15; A61F 13/20

(52) U.S. Cl. 604/358

(57) ABSTRACT

A semi-rigid waist, posterior, and thigh enclosing garment used as a diaper is worn by an individual unable to control their bodily functions. The seat portion of the garment is compartmentalized to allow for the collection of feces and urine. The garment is preferably elastic around the thighs and waist to allow for a sufficient seal for the containment of waste, waste flushing water, and spray cleaning water. The garment is also preferably primarily made of silicone. The upper portion of the garment has an elongated oval opening leading to a lower, waste-collecting chamber. The waste-collecting chamber holds the waste away from the individual's body. A raised, pliable ring is positioned to both surround the elongated oval opening and ring the rectum and urethra of the wearer. The garment is preferably connected to a portable cleaning unit or is removed from the wearer for cleaning in a separate device.

### BACKGROUND OF THE INVENTION

1. Field of the Invention

The invention pertains to the field of diaperlike garments. More particularly, the invention pertains to the field of sanitary devices for individuals who are incontinent.

2. Description of Related Art

Diaperlike garments have traditionally been provided for both adults and children. Typically, these garments contain absorptive materials for absorbing waste. These diapers must be periodically changed, especially when they become soiled or can absorb no more moisture.

The changing process is especially difficult for people who are confined to a wheelchair or have difficulty walking. One attempt to solve this problem is a diaper cup which surrounds a user's hips and buttocks, disclosed in U.S. Pat. No. 5,681,927, issued Oct. 28, 1997, and U.S. Pat. No. 6,110,159, issued Aug. 29, 2000, both entitled “DEVICE FOR DISPOSING EXCREMENT”. A system of hoses and suction wash the diaper cup while it is being worn by a user. However, these complicated devices do not have a waste-collecting chamber to effectively collect waste away from the user.

Another attempt to solve this problem includes U.S. Pat. No. 5,944,338, entitled “WHEELCHAIR”, issued Aug. 31, 1999. The wheelchair in this patent includes a toilet assembly which fits into the seat of the wheelchair. Although the bowl portion of the toilet assembly may be removed without the user getting up, adding the toilet assembly to the chair requires the seat portion of the wheelchair to be detached from the wheelchair. U.S. Pat. No. 4,795,214, entitled “CONVERTIBLE WHEELCHAIR/LITTER”, issued Jan. 3, 1989, similarly adds a toilet seat assembly to a wheelchair. Both of these patents require a toilet assembly which is part of a wheelchair. There is also no real containment of the waste, except in a common receptacle. In addition, neither patent provides any type of self-cleaning associated with the toilet assemblies. Someone must come, remove, and replace the bowls in the toilet seat assemblies of each of these wheelchairs when they are dirty.

Therefore, there is a need in the art for a garment which provides nonabsorptive containment of waste, which can also be cleaned and reused, preferably without removing the garment from the wearer.

### SUMMARY OF THE INVENTION

A semi-rigid waist, posterior, and thigh enclosing garment used as a diaper is worn by an individual unable to control their bodily functions. The seat portion of the garment is compartmentalized to allow for the collection of feces and urine. The garment is preferably elastic around the thighs and waist to allow for a sufficient seal for the containment of waste, waste flushing water, and spray cleaning water. The garment is also preferably primarily made of silicone. The upper portion of the garment has an elongated oval opening leading to a lower, waste-collecting chamber. The waste-collecting chamber holds the waste away from the individual's body. A raised, pliable ring is positioned to both surround the elongated oval opening and ring the rectum and urethra of the wearer.

In a preferred embodiment, the garment is connected to a portable cleaning unit. In one embodiment, the portable cleaning unit preferably includes a water holding tank, a collection tank, a compressor, a water pump, a power source, and a waste holding tank. Water from the water holding tank is directed into and out of the lower, waste-collecting chamber to flush the waste through the collection tank and into the waste holding tank. The waste holding tank is preferably equipped with an inlet and outlet for the purpose of mating to a stationary docking unit which directs water into the portable cleaning unit, and flushes accumulated waste out of the portable cleaning unit and into the sewage system. In an alternative embodiment, the portable cleaning unit is a two chambered box with inlets and outlets for the flow of water. A system for absorbing and neutralizing odors is also provided.

### BRIEF DESCRIPTION OF THE DRAWINGS

FIG. 1 shows a top view of the garment portion of the apparatus in an embodiment of the invention.

FIG. 2 shows a bottom view of the garment portion of the apparatus in an embodiment of the invention.

FIG. 3 shows a cross-sectional view of the garment portion of the apparatus in an embodiment of the invention.

FIG. 4 shows a cross-sectional side view of the garment portion of the apparatus in an embodiment of the invention.

FIG. 5 shows a view of the garment portion mated with a portable cleaning unit portion of the apparatus in an embodiment of the present invention.

FIG. 6 shows the apparatus of the invention mated with a wheelchair in an embodiment of the present invention.

FIG. 7 shows the apparatus of the invention mated with a wheelchair where the portable cleaning unit is extended behind the wheelchair in an embodiment of the present invention.

FIG. 8A shows a schematic drawing of a garment in an embodiment of the present invention.

FIG. 8B shows a cross-sectional view taken along line A-A of the garment shown in FIG. 8A.

FIG. 8C shows another schematic drawing of the garment shown in FIG. 8A.

FIG. 8D shows a cross-sectional view taken along line B-B of the garment shown in FIG. 8C.

FIG. 9 shows a garment having successive layers of silicone each having a particular shape in an embodiment of the present invention.

FIG. 10 shows a blown up expanded view of the garment shown in FIG. 9.

FIG. 11 shows a close up view of the holes shown in the garment of FIGS. 9 and 10.

FIG. 12 shows a wheelchair containing odor-absorbing flaps in an embodiment of the present invention.

FIG. 13 shows a close-up view of the flaps shown in FIG. 12.

FIG. 14 shows a back view of the wheelchair shown in FIG. 12.

FIG. 15 shows a close-up view of the flap and piping which absorbs odor from the garment in the embodiment of the invention shown in FIG. 12.

FIG. 16 shows a close-up view of the portable cleaning unit shown in FIG. 12.

FIG. 17 shows a schematic drawing of a device for cleaning the garment in a preferred embodiment of the invention.

FIG. 18 shows a close-up view of the inside of the portable cleaning unit shown in FIG. 16.

### DETAILED DESCRIPTION OF THE INVENTION

The garment of the present invention provides nonabsorptive containment of waste. The garment has a wearable, non-absorptive body having a lower, semi-rigid, waste-collecting chamber for collecting feces, urine, and water away from a wearer during use. The garment is designed to fit males or females interchangeably for the purpose of waste entry. Light duty pressure release valves account for the displacement during waste entry. A waste entryway has a raised ring around its perimeter to aid in creating a seal, enhanced by the wearer's weight. The necessary seal around all garment openings may also be aided through the employment of inflatable air pockets, producing a tolerable yet firm pressure when needed.

In a preferred embodiment, silica pellets are strategically placed to absorb odors incident to the garment's use. In one embodiment, these silica pellets are placed in one or more compartments around the waist of the wearer, or in other locations within the garment, and aid in the absorption of noxious odors, as airflow is channeled through these silica-containing pockets. This silica “belt” is preferably removable from the rest of the garment to aid in cleaning the garment. This configuration also flushes out odors from the silica using airflow, essentially recharging the silica. Alternatively, odor-absorbing silica pellets are incorporated into a flap system which attaches to a wheelchair in which the garment wearer is confined. The flaps, entirely separate from the garment, act to absorb odor and pull it away from the wearer during use.

In one embodiment, the garment is used on wheelchair-bound adults. In this embodiment, the inlets and outlets for flushing the waste are attached while on the wearer. Additionally, the garment preferably has inlets for fresh air to be periodically forced through to guard against the type of skin problems associated with prolonged sitting or being confined to a bed.

In an alternative embodiment, the garment is used on a baby. The garment is preferably made as described above. The garment's function differs from that of a conventional diaper in the same way as the garment meant for wheelchair bound adults in that it has a void to collect waste away from the wearer's skin instead of an absorbent material. In addition, it has the ability to direct odor laden air through odor absorbent silica before being released to the environment. In this embodiment, a separate device is preferably employed for cleaning.

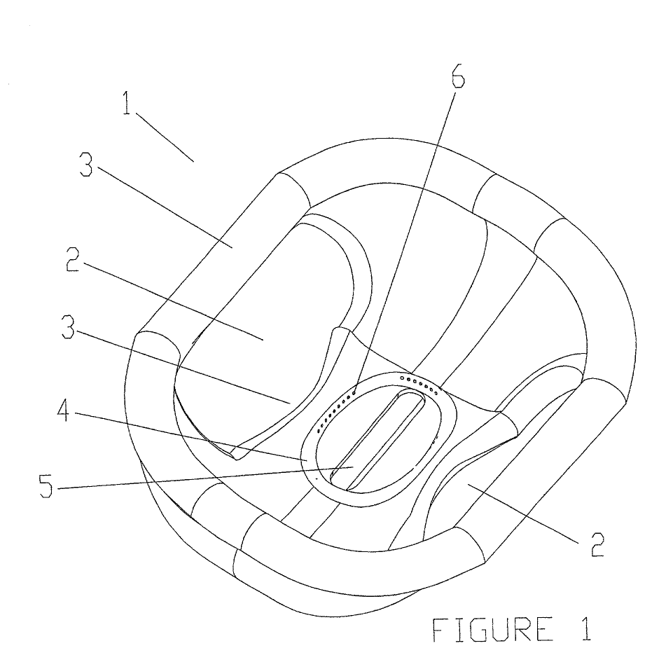

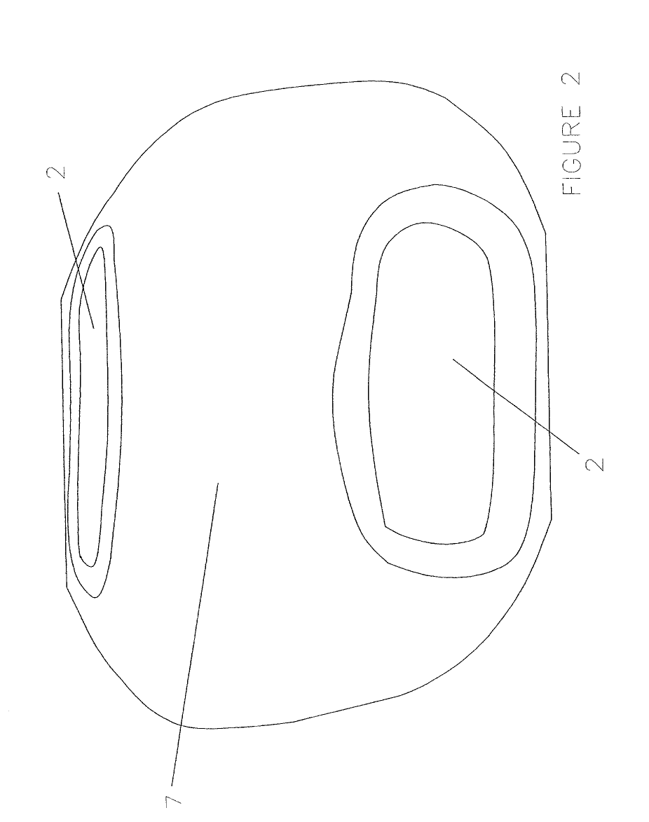

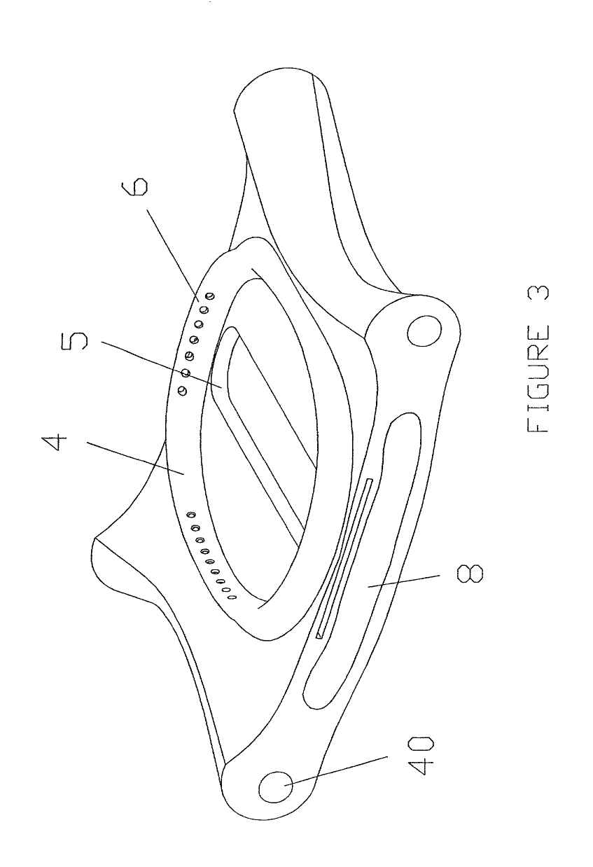

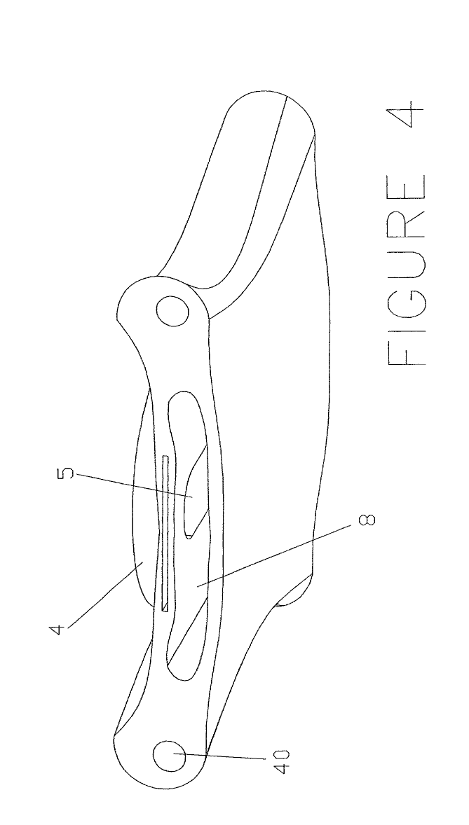

Referring now to FIGS. 1 through 4, one example of the garment (1) is shown. The garment (1) is preferably composed of a semi-rigid, waterproof material, such as silicone. There are two holes (2) in the garment (1) for the placement of a wearer's legs during use. The shape of the bottom (7) of the garment (1) conforms to an individual's thigh and buttocks region. The garment preferably has a band (3) around the thighs and waist. The band (3) creates a seal for the containment of waste, waste flushing water, and spray cleaning water and prevents the garment (1) from leaking. In a preferred embodiment, a hollow ring (40) is built into the garment around the perimeter of the leg holes (2). This hollow ring (40) is preferably slightly inflatable to aid in sealing the garment (1). A preferable material to use as the band (3) is an elastic material, although any material which successfully creates a seal around the wearer could be used.

Silica pellets are preferably placed in one or more compartments in the band (3) around the waist of the wearer to aid in the absorption of noxious odors, as airflow is channeled through these silica-containing pockets. This silica “belt” is preferably removable from the rest of the garment to aid in cleaning the garment. Silica pellets are alternatively placed in other locations within the garment.

The seat portion of the garment is compartmentalized to allow for the collection of feces and urine in a lower, waste-collecting chamber (8). The lower, waste-collecting chamber (8) collects feces, urine and water away from the wearer during use. The upper portion of the garment (1) has an elongated oval opening (5) leading to the lower, waste-collecting chamber (8). The elongated oval opening (5) is positioned to be generally under the rectum and urethra of the wearer during use. A raised, pliable ring (4) is positioned to both surround the elongated oval opening (5) and ring the rectum and urethra of the wearer. The ring (4) serves the dual purpose of directing waste into the lower, waste-collecting chamber (8), while also serving as a conduit to direct a water spray to the wearer's rectum to aide in cleaning their rectum after waste has been deposited. Water is directed into the garment (1) through water dispensing holes (6) located on the inner sides of the raised, pliable ring (4). Water is preferably directed into the holes through an inlet valve.

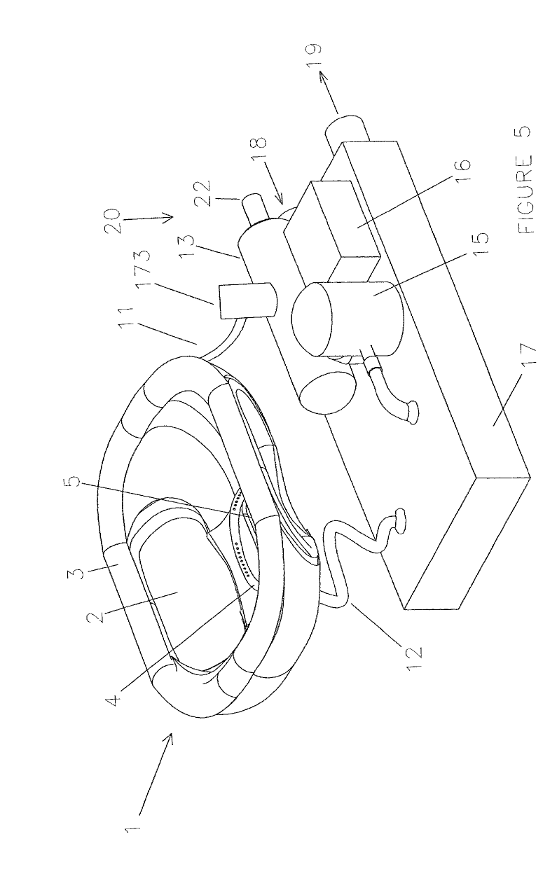

Referring also to FIG. 5, a portable cleaning unit (20) connects to at least the lower, waste-collecting chamber (8) of the garment. The portable cleaning unit (20) is preferably connected to the garment (1) via piping (11) and (12). The piping (11) and (12) is preferably made of a flexible material to allow for flexibility in the connection between the garment (1) and the portable cleaning unit (20). Some examples of materials to use for the piping (11) and (12) include, but are not limited to, plastic, rubber, or any other flexible material. The garment (1) preferably connects to the portable cleaning unit (20) via a fitting on the underside of the garment. For example, a removable coupling could be used. The piping (11) leads from water holding tank (13) and acts as a water supply line for the garment (1). The water is preferably supplied to the garment (1) via a water pump (173). If the wearer is capable of initiating the cleaning cycle herself, she turns a switch or button whenever she determines cleaning is needed. Alternatively, if the wearer is immobile, another individual could turn the switch or button when needed. In a third example, a moisture sensitive apparatus is incorporated into the garment (1). In this embodiment, a cleaning cycle is initiated when the moisture-sensitive apparatus detects a certain level of moisture.

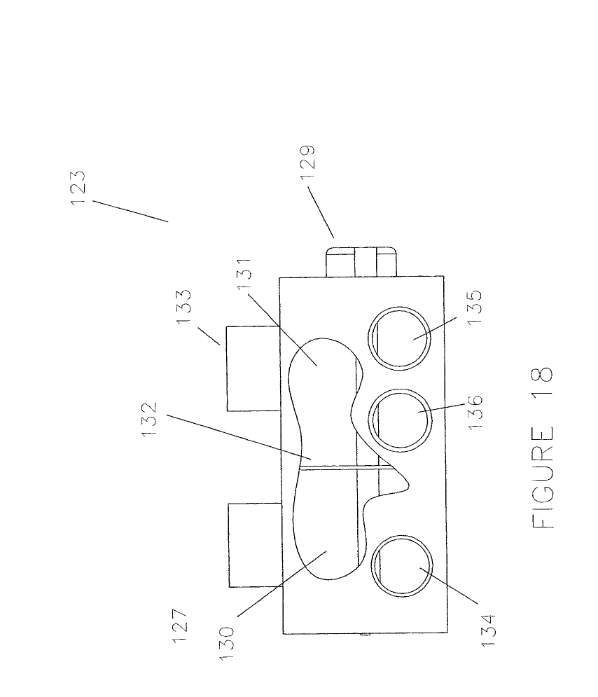

Water from a water holding tank (13) is directed into and out of the lower, waste-collecting chamber (8) to flush the waste into a collection tank (17). The water is dispensed through the water dispensing holes (6) to clean out the garment (1). Once dispensed through the water dispensing holes (6), the water, as well as any residual waste, moves into the lower, waste-collecting chamber (8) of the garment (1). At this time, the piping (12) acts as a waste supply line. Dirty water and any feces or urine fall by gravity (and a small vacuum if an alternative portable cleaning unit (123), see FIGS. 16 and 18, is used) through piping (12) into the collection tank (17). The water pump (173) directs water for the flushing out of accumulated waste within the garment (1) from the water holding tank (13) through the lower, waste-collecting chamber (8) into the collection tank (17). The collection tank (17) is provided with a compressor (15) which directs displaced air as well as accumulated and stray waste gasses into the collection tank (17). A power source (16) is provided to run the compressor (15) and water pump (173). The power source (16) is preferably a rechargeable battery.

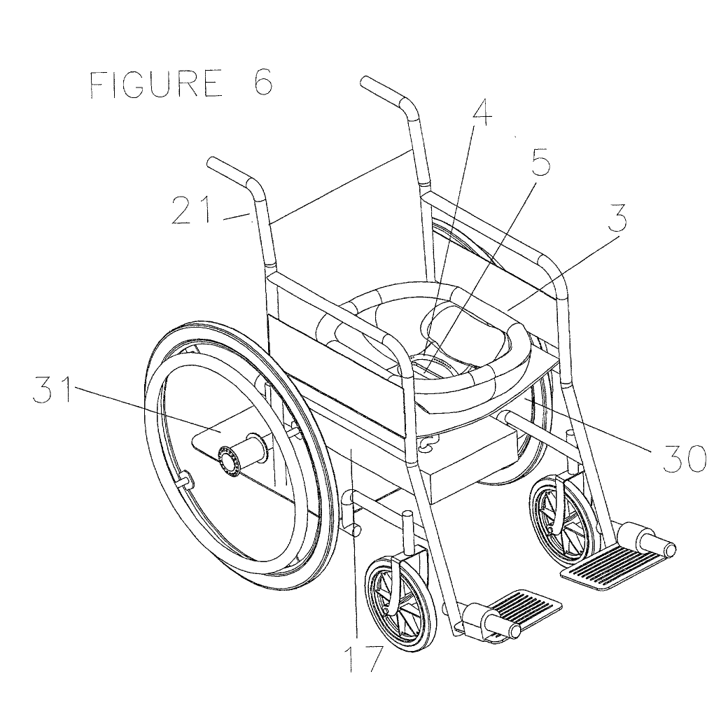

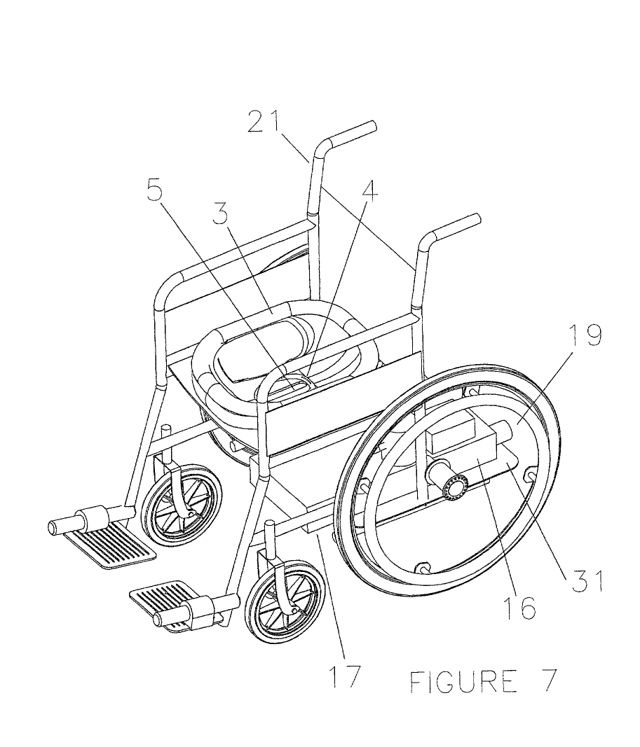

Referring also to FIGS. 6 and 7, in a preferred embodiment of the invention, the garment (1) and portable cleaning unit (20) are used in concert with a wheelchair (21). An individual (not shown) wearing the garment (1) sits in the seat (30) of the wheelchair (21), while the portable cleaning unit (20) sits on a shelf (31) below the seat (30) of the wheelchair (21). In this embodiment, the wearer (not shown) is able to move around in the wheelchair (21) while still being afforded the convenience of the cleanliness given when he wears the garment (1).

The portable cleaning unit (20) is preferably equipped with a water inlet (18) and a sewage outlet (19) for the purpose of mating to a stationary docking unit which directs water into a waste container, flushing accumulated waste out of the container and into the sewage system. The stationary docking unit preferably includes an incoming water line and a waste line for the portable cleaning unit (20). The inlet (18) and outlet (19) are provided with opening and closure mechanisms which either simultaneously open or simultaneously close, or seal, the inlet (18) and outlet (19). This unique arrangement allows for the collection of waste and the cleaning of the garment (1) and wearer at any remote location from the stationary docking unit which cleans the portable cleaning unit (20).

In addition to the collection tank (17) mating with the water inlet (18) and the sewage outlet (19), the water holding tank (13), equipped with a displaced air escape valve, is fitted with an automatically opening and closing inlet valve (22) which mates with the supply line water to refill the water holding tank (13).

The stationary docking unit performs multiple functions. It provides clean water to the water holding tank (13) by connecting to the valve (22). The docking unit also connects to inlet (18) and outlet (19). The docking unit provides water through the inlet (18) to clean out the waste holding tank (17). In addition, it removes waste from the waste holding tank (17) through outlet (19). By having the docking unit separate from the apparatus of the present invention, the wearer is permitted to move around in a wheelchair, or lie in their bed, without being restricted by connection to a stationary unit. Since the stationary docking unit is not connected to the apparatus at all times, the wearer is not restricted by the apparatus. The garment has effectively removed the need for a conventional toilet in the care of incontinent individuals.

The wheelchair embodiment, discussed above, is especially convenient to wheel the portable cleaning unit (20) up to the stationary docking unit. Specifically, the inlet (18) and the outlet (19) on the portable cleaning unit (20), as well as water line inlet (22) are easily connected to the conventional plumbing (fresh water and waste line) which encompass the stationary docking unit due to their location on the wheelchair (21).

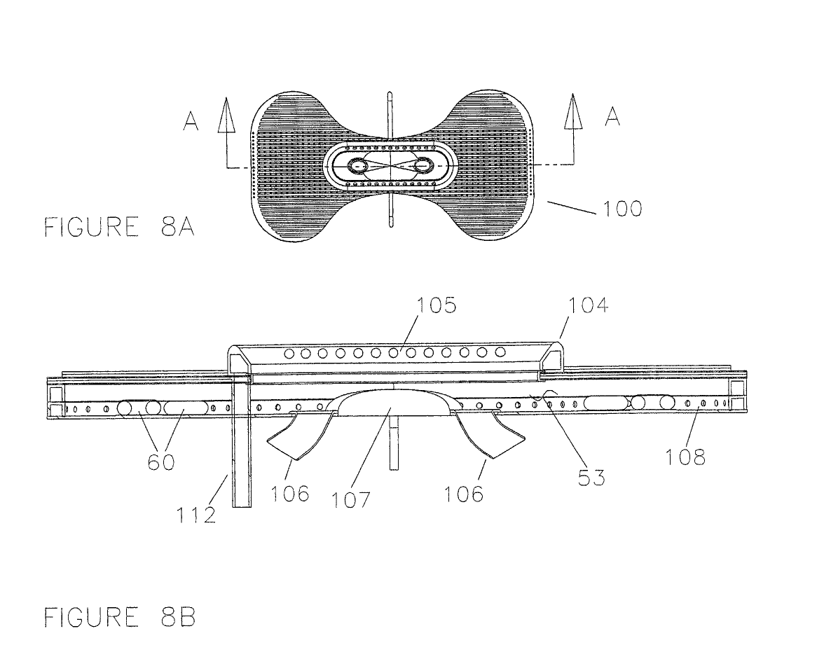

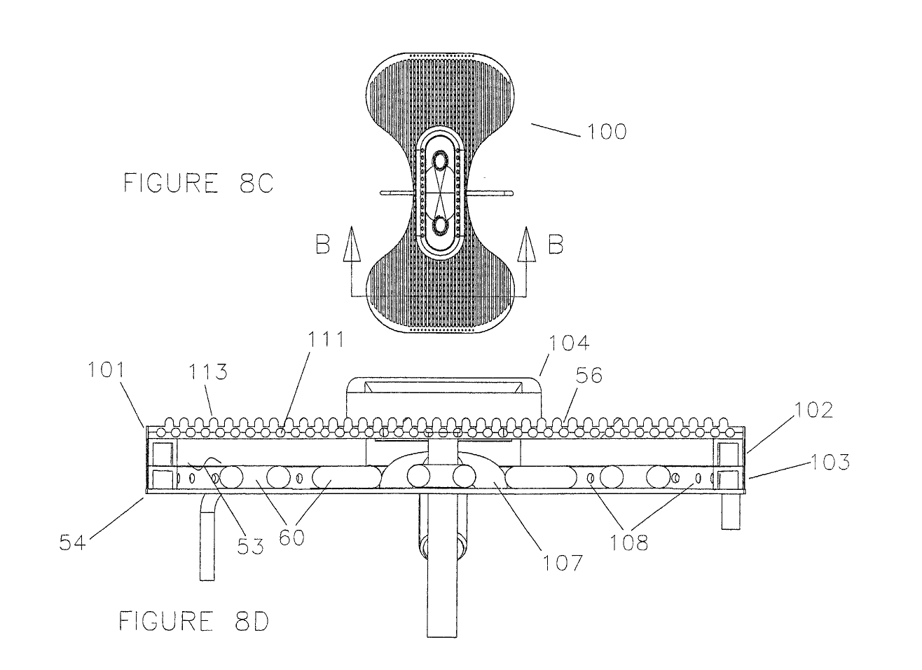

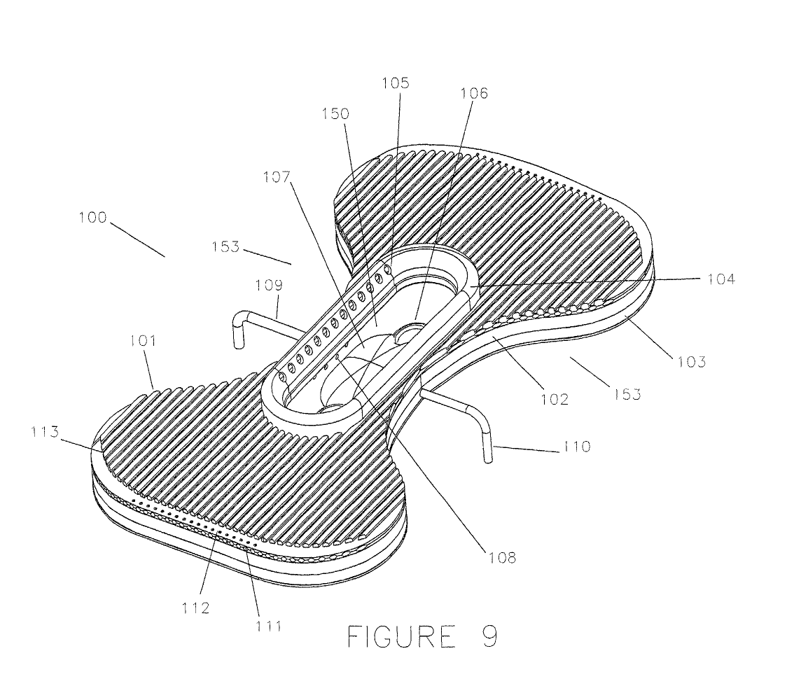

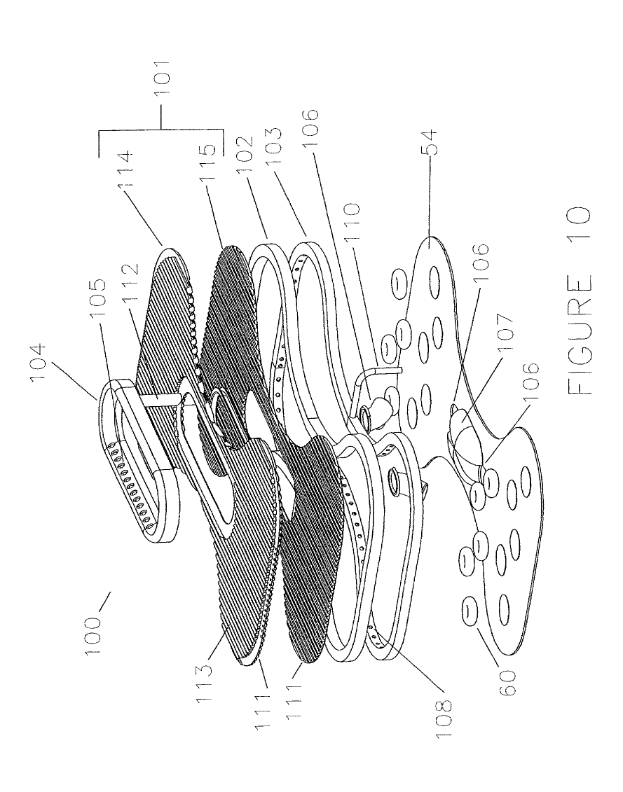

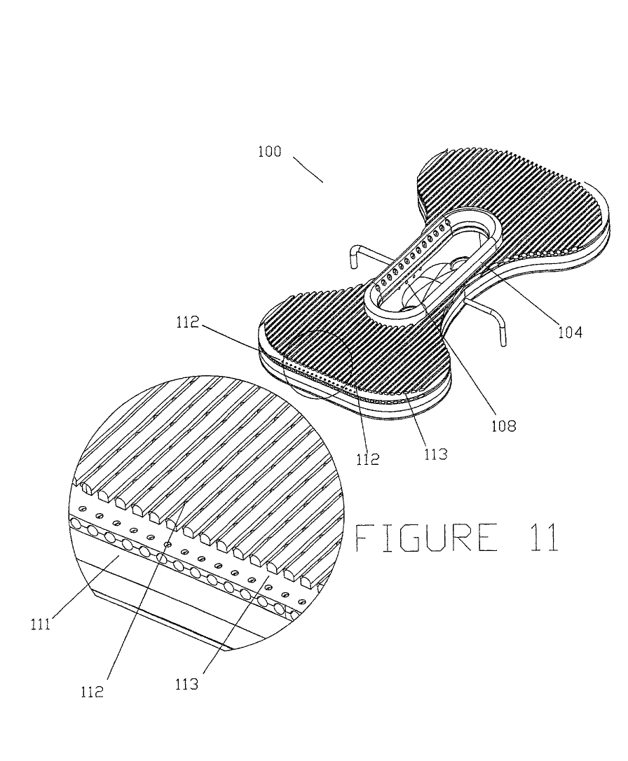

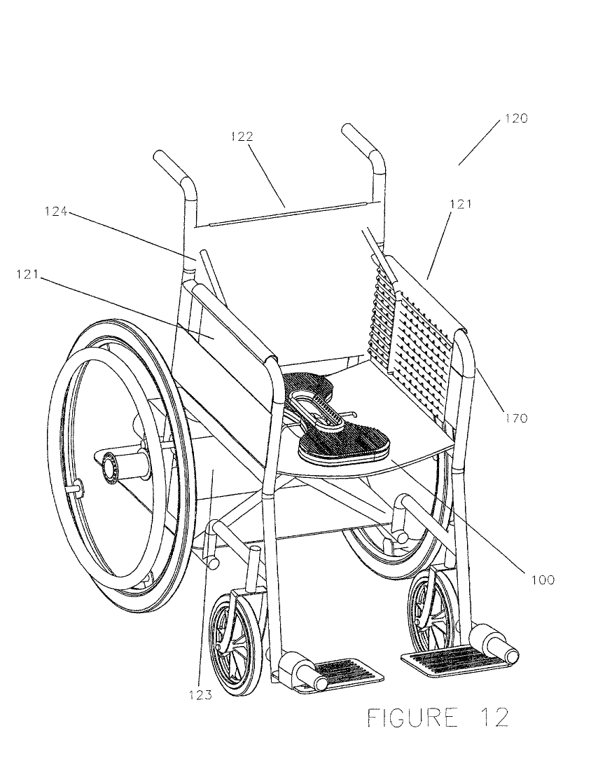

A multi-layered, compartmentalized garment (100) is shown in FIGS. 8A through 11. Although the garment (100) is flat in these figures, its shape creates spaces (153) for the placement of a wearer's legs during use. The garment's hourglass or other diaper like shape allows it to be fastened around a user's legs. Sample fasteners include, but are not limited to, buttons, snaps, hooks and eyes, hook and loop tape (e.g.—Velcro®), or similar materials. The garment (100) has multiple layers (101), (102), (103), and (54), each preferably constructed of silicone. The silicone is preferably in the form of sheets, donuts (60), balls, or other shapes. Although four layers (101), (102), (103), and (54) are shown in these figures, any number of layers could be used. The figures exemplify different types of layers which functionally enhance the garment (100). The garment, using layers (101), (102), (103), and (54), forms an envelope of sorts, which when sat upon does not collapse due to the solid, air or liquid filled silicone shapes (60) at appropriate locations to support the weight of the wearer sufficiently to maintain the voids (53) between layers (102) and (103) of the garment.

Layer (101) is the layer closest to a wearer's skin during use. This layer (101) preferably has ridges (113) which touch the skin. These ridges (113) perform a dual function. First, they aid airflow by allowing air to penetrate between the ridges (113). In addition, since the entire inside of the garment is no longer contacting the skin, they prevent chafing of the skin during use. Holes (112), which allow air flow, are preferably located between the ridges (113). Additional holes (111), which preferably run through the entire garment (100), are perpendicular to and meet up with holes (112). There are also preferably air inlets (56) built into the layer (101) of the garment. These air inlets (56) also aid air flow into the garment. Layer (101) is shown in two parts (114) and (115) in FIG. 10 to allow a better view of layer (101). Layer (101) is not necessarily two layers, although two layers are possible in a preferred embodiment. The ridges (113) are on the top of partial layer (114). Half of the holes (111) are on the bottom of partial layer (114) and the other half of the holes (111) are on the top of partial layer (115).

An inflatable perimeter member (102) is preferably directly below layer (101). An air inlet (109) provides air to this member (102) for inflation. When the inflatable member (102) is inflated, it opens up the garment (100) and makes it more effective in accepting waste during use. Although only a perimeter of the inflatable member (102) is shown in the figures, the entire inflatable member (102) could vary in shape and be located anywhere it is needed. The lower member (103) preferably has inward-facing holes (108) around its perimeter. These holes (108) provide entry points for water which has entered through a water inlet (110). The lower member (103) allows water to be sprayed throughout the garment (100). This perimeter member (103) is preferably a thin layer in which water can be sprayed from the holes (108) in the perimeter. The outer layer (54) of the garment (100), the layer farthest away from the wearer's skin, preferably has silicone donuts (60) or other silicone shapes built into it to give it structure. Although the donuts (60) shown in the drawing are detachable, this is merely a schematic drawing and the donuts (60) are preferably built into the outer layer (54) of the garment (100).

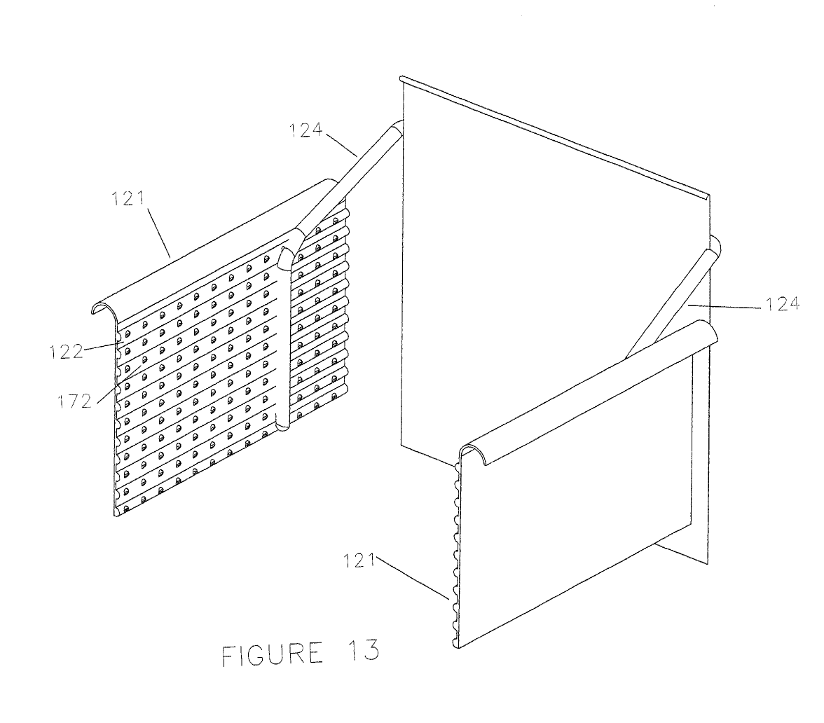

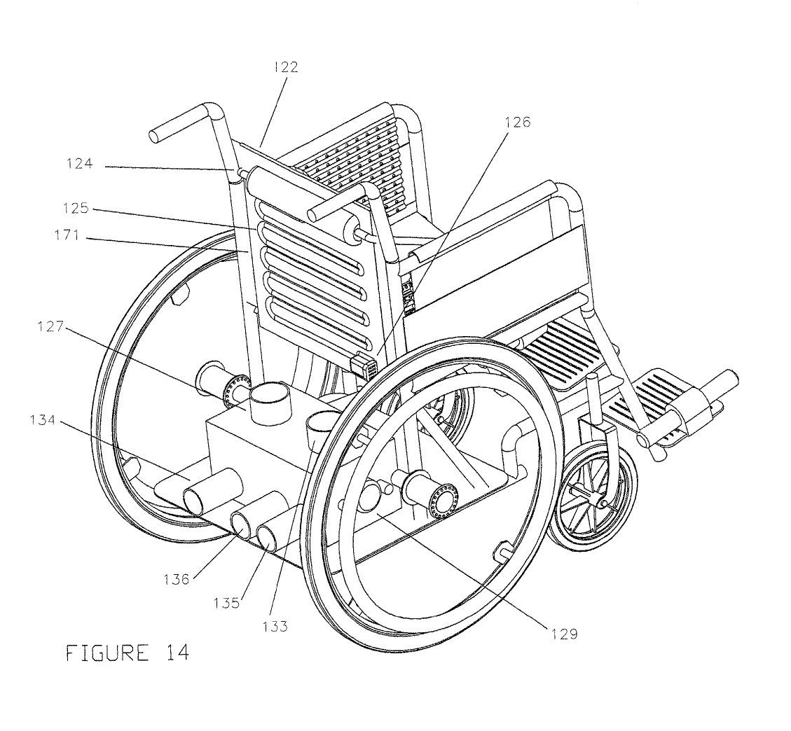

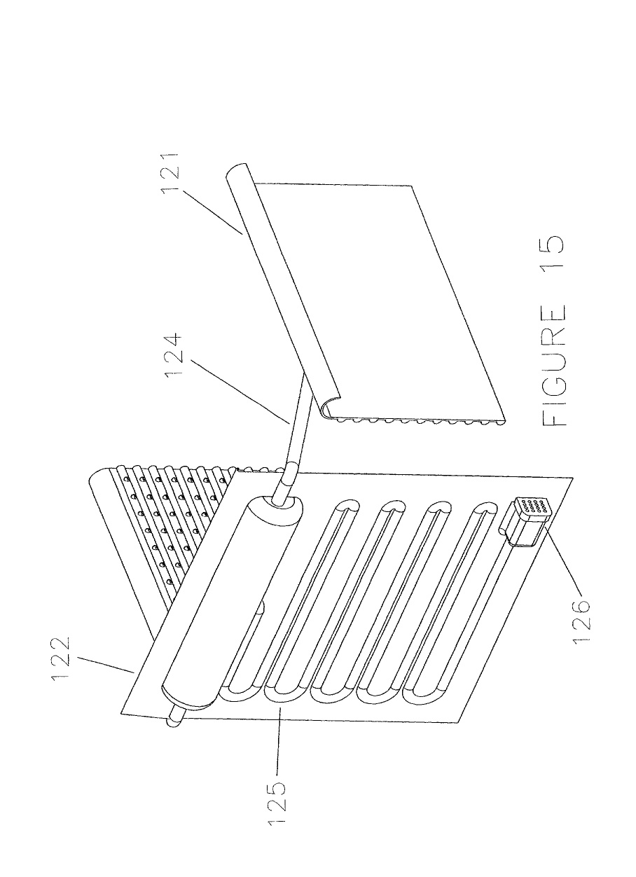

A lower, waste collecting chamber (150) is preferably semi-rigid, and collects feces, urine and water away from a wearer during use. A raised ring (104), similar to the raised ring (4) shown in FIGS. 1 through 4, is located substantially directly below a rectum and urethra of a wearer during use. The ring (104) serves the dual purpose of directing waste into the lower, waste-collecting chamber (150), while also serving as a conduit to direct a water spray to the wearer's rectum to aide in cleaning their rectum after waste has been deposited. Holes (105), which direct the water spray into the garment (100) when it is soiled, are preferably located on an inside perimeter of the raised ring (104). An inlet hose (112) in the raised ring (104) provides water to the cleaning area. In a preferred embodiment, spraying happens sequentially. Outlets (106), which are built into the garment (100), transfer waste, urine and dirty water to a portable cleaning unit (20) or (123) (discussed below, see FIGS. 12, 14, and 16). In a preferred embodiment, a raised area (107) rests on the wearer's perineum. This raised area (107) provides separation of the areas directly below urinary and bowel functions, adding a high degree of cleanliness to the device. Any of the features described with respect to garment (1), above, could be substituted for the features described with reference to garment (100), and vice versa, without deviating from the spirit of the invention.

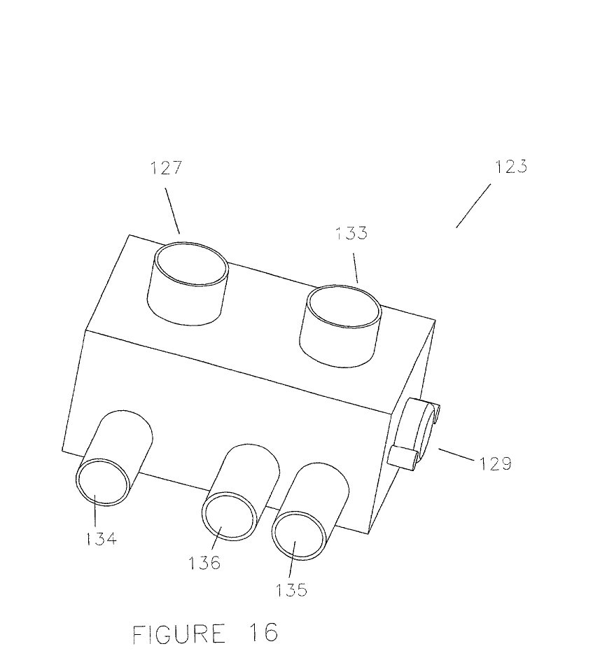

Referring also to FIGS. 12 through 16 and 18, in a preferred embodiment of the invention, the garment (100) and a portable cleaning unit (123) are used in concert with a wheelchair (120). An apparatus comprising the portable cleaning unit (123) and the garment (100), or even the garment (100) alone, is also usable without the wheelchair (120). An individual (not shown) wearing the garment (100) sits in the wheelchair (120), while the portable cleaning unit (123) sits on a shelf (151). In this embodiment, the wearer (not shown) is able to move around in the wheelchair (120) while still being afforded the convenience of the cleanliness given when she wears the garment (100).

The garment (100) attaches to the portable cleaning unit (123) or a portable cleaning unit (20) as described above. Portable cleaning unit (123) is a two chambered, (130) and (131), box with a wall (132) between the two chambers (130) and (131). A motor (129) moves the wall (132) between the chambers (130) and (131) of the portable cleaning unit (123). This portable cleaning unit (123) could also be used with the garment (1) shown in FIGS. 1 through 4. In addition, the garment (100) described in FIGS. 8A through 11 could be used with portable cleaning unit (20).

A water inlet (127) in the portable cleaning unit (123) is preferably attached to the water inlet (110) of the garment (100). This allows the portable cleaning unit (123) to provide the garment (100) with water for cleaning the garment (100). When the garment (100) needs to be cleaned, the motor (129) is attached to an actuator, which provides piston-like movement of the wall (132). The motor (129) pushes the wall (132) farther into the clean rinse water compartment (130). This action causes compression in the chamber (130), forcing clean water out of the portable cleaning unit (123), through the outlet (127), and into the garment (100). It also simultaneously creates a vacuum in the chamber (131) for collecting waste. Thus, the garment is rinsed and cleaned by the supply of clean water. At the same time, dirty water and waste are pulled out of the garment (100) through the outlets (106) in the garment and into an inlet (133) for dirty water in the portable cleaning unit (123). In one embodiment, the motor (129) is manually activated. Alternatively, the motor (129) is activated upon sensing waste in the garment. For example, optoelectronic or ultrasonic level sensors could be used. Upon a threshold change in the optical or acoustic properties of the lower, waste collecting chamber (150), respectively, the motor is activated and the cleaning process begins.

When the portable cleaning unit (123) itself needs cleaning and replenishment of rinse water, attachment to a fresh water system is provided through a connector (134) on the clean water reservoir (130). Simultaneous connection to the sewage system is provided for the waste container (131) by connector (136). Rinse water is provided to afford effective cleaning of the waste chamber by connector (135). This arrangement provides action similar to flushing of a toilet to clean the portable cleaning unit and prepare it for another period of service.

These figures also show a system for absorbing odors incident to the garment's (100) use. An odor collector has side flaps (121) which preferably fit on the arms (170) of a wheelchair (120). A back flap (122) preferably fits on the back of the wheelchair. Although flaps (121) and back flap (122) are attached to a wheelchair (120) in this embodiment, they are removable from the wheelchair (120) and can be used in any location where the garment (100) user may be located. In addition, a single flap (121) or additional flaps (121) in other locations on the wheelchair (120) can also substitute for the two flaps (121) of the odor collector shown in the figures. The flaps (121) and (122) are preferably pieces of flexible material, for example rubber, which hang on the sides (170) and back (171) of the wheelchair (120).

The side flaps (121) preferably have rows (122) of holes (136). The flaps (121) catch odors in the air through these holes (172). Although rows (122) of holes (136) are shown in the figures, any configuration of holes (172) which catch odors in the air is within the scope of the invention. A fan (126) on the back flap (122) pulls the odor-laden air from the side flaps (121) through piping (124) connecting the side flaps (121) and the back flap (122). Once the air is brought to the back flap (122), it passes through additional piping (125). The piping (125) is filled with silica pellets. The silica pellets absorb the odors as the air passes through the piping (125). The piping (125) can be in any configuration which allows sufficient surface area for the silica pellets to absorb odors. In this system, odor-laden air manifolds together and is passed through a path of silica pellets to absorb the odors.

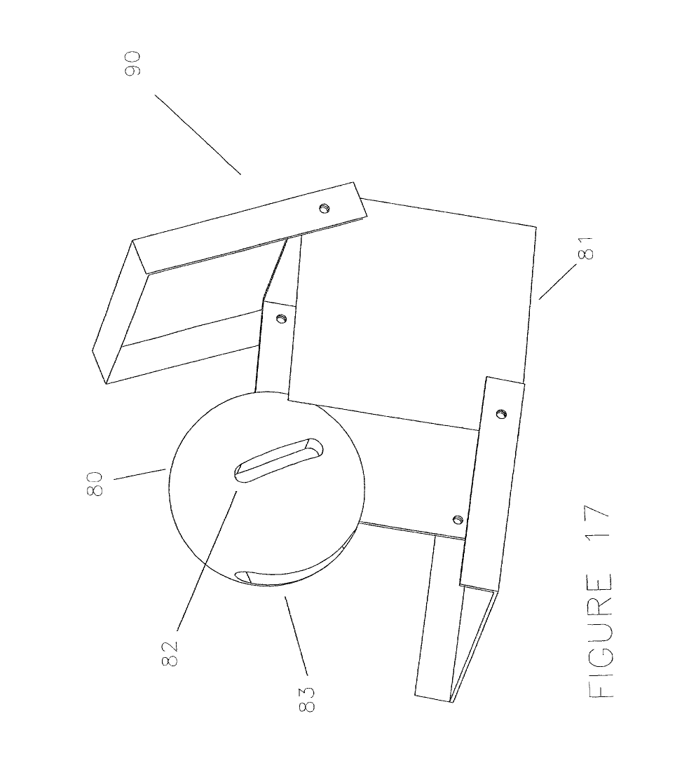

In an embodiment where the garment (1) or (100) is taken off the wearer before cleaning, a separate device is employed for cleaning. An example of such a device is shown in FIG. 17. After removing a waste-filled garment from a baby, the garment is placed on a ball-shaped form (80) which is approximately the same shape as a baby's bottom. In an alternative embodiment, the ball-shaped form (80) is sized for an adult who has used the garment. This ball-shaped form (80) allows the garment to retain its shape against collapse, and preferably fits into a small, table top box, or cleaning unit (81). Although a ball-shaped form (80) is shown here, any form which adequately supports the garment against collapse could be used. The ball-shaped form (80) preferably has a hole (83) which approximates the elongated oval opening (5) of the garment, so that the waste from a dirty garment is transferred to the hole (83) in the ball-shaped form (80) during cleaning. In addition, the mating of these two openings (5) and (83) allow for efficient cleaning of the garment by the cleaning device (90).

In a preferred embodiment, when the cleaning unit (81) is opened, the ball-shaped form (80) comes slightly out of the cleaning unit (81) so it is easily accessible. In addition, it rotates so that the hole (83) is on the bottom of the ball-shaped form (80). This allows the garment to be placed on the ball-shaped form (80) without any of the waste being spilled. The ball-shaped form (80) retracts back into the cleaning unit (81) after the garment is placed upon it. When the cleaning unit (81) closes, the ball-shaped form (80) preferably rotates until the hole (83) is located on the top of the ball-shaped form (80). Gravity helps the waste out of the garment and into the hole (83) for cleaning.

The ball-shaped form (80) has appropriately placed water inlets and outlets (82) for the purpose of allowing clean water into the garment and flushing waste out of the garment, respectively. Although one inlet/outlet (82) is shown here, multiple inlets and outlets (82) are preferably placed in the ball-shaped form (80) to aid in cleaning. The cleaning unit (81) preferably connects to hard-wired plumbing which supplies clean water and removes dirty water and feces from the garment. The cleaning device (90) has the capacity to clean, sterilize (with heat and/or disinfectants), and finally air dry the garment, quickly readying it for another use.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An apparatus for disposal of human waste comprising a garment comprising a wearable, non-absorptive body having a lower, semi-rigid, waste collecting chamber for collecting feces, urine, and water away from a wearer during use.

2. The apparatus of claim 1, further comprising a portable cleaning unit which connects to at least the lower, waste collecting chamber of the garment, wherein the portable cleaning unit comprises a water supply line which distributes water to the garment and a waste supply line which takes water and waste away from the garment.

3. The apparatus of claim 2, wherein the apparatus is removably mounted to a wheelchair.

4. The apparatus of claim 2, wherein the garment further comprises:
a) an elongated raised ring, having a hole located directly above the waste-collecting chamber wherein the hole is located substantially directly below a rectum and a urethra of a wearer during use; and
b) a plurality of water dispensing holes located on an inside perimeter of the ring connected to the water supply line of the portable cleaning unit, such that water is directed into the garment to clean the wearer after a deposition of waste.

5. The apparatus of claim 4, wherein the garment further comprises a band around a top perimeter of the garment such that the band provides a seal for the garment.

6. The apparatus of claim 2, wherein the portable cleaning unit further comprises:
a) a water holding tank which connects to the water supply line; and
b) a collection tank which connects to the waste supply line to collect waste and dirty water from the garment.

7. The apparatus of claim 6, wherein the portable cleaning unit further comprises a water pump to pump water through the water inlet line.

8. The apparatus of claim 7, wherein the portable cleaning unit further comprises a battery.

9. The apparatus of claim 6, wherein the portable cleaning unit further comprises:
c) an inlet for adding clean water to the portable cleaning unit; and
d) an outlet for disposing of dirty water and waste in the portable cleaning unit.

10. The apparatus of claim 9, wherein the inlet and the outlet are simultaneously open or closed.

11. The apparatus of claim 9, further comprising a docking unit which connects to the inlet to pump water into the portable cleaning unit, and connects to the outlet to pump waste out of the portable cleaning unit.

12. The apparatus of claim 2, wherein the portable cleaning unit comprises a box having an interior divided into a first chamber a and a second chamber by a movable wall;
wherein the wall moves in a first direction to increase an amount of space inside the first chamber and consequently decrease an amount of space inside the second chamber, or alternatively in a second direction to decrease the amount of space of the first chamber and consequently increase the amount of space inside the second chamber; and
wherein the first chamber connects to the water supply line and the second chamber connects to the waste supply line.

13. The apparatus of claim 12, wherein the portable cleaning unit further comprises a motor attached to an actuator for moving the wall in the first direction.

14. The apparatus of claim 1, wherein the garment is made of silicone.

15. The apparatus of claim 14, wherein the garment further comprises a plurality of layers of silicone, such that a shape of the layers forms the lower, waste collecting chamber.

16. The apparatus of claim 1, further comprising a plurality of silica pellets placed within the garment, wherein the silica pellets absorb odors.

17. The apparatus of claim 16, wherein the garment further comprises a belt which fits around a waist of the wearer when the apparatus is in use, wherein the silica pellets are located within the belt.

18. The apparatus of claim 17, wherein the belt is removable from the garment.

19. The apparatus of claim 1, wherein the garment further comprises an elongated raised ring, having a hole located directly above the waste-collecting chamber wherein the hole is located substantially directly below a rectum and a urethra of a wearer during use; and the apparatus further comprises a cleaning device upon which the garment is placed after removal from the wearer, comprising:
a) a ball-shaped form which allows the garment to retain its shape, wherein the ball-shaped form comprises:
i) at least one inlet to receive clean water;
ii) at least one outlet to flush out the garment; and
iii) an elongated opening approximately a same shape as the hole in the elongated raised ring such that the elongated opening mates with the hole during cleaning; and
b) a housing unit for the ball-shaped form, wherein the housing unit provides plumbing to the ball-shaped form.

20. The apparatus of claim 1, wherein the garment further comprises a plurality of air inlets built into a skin-facing side of the garment during use, such that the air inlets provide air to the garment.

21. The apparatus of claim 1, wherein the garment further comprises a plurality of ridges located on a skin-facing side of the garment during use.

22. The apparatus of claim 1, wherein the garment further comprises a system of piping throughout the garment, which distributes water throughout the garment during cleaning.

23. The apparatus of claim 1, wherein the garment further comprises a raised area located in the lower, waste collecting chamber such that the raised area rests on a wearer's perineum during use, separating urinary and bowel functions.

24. The apparatus of claim 1, further comprising:
a) an odor-absorbing system for absorbing odors created by a use of the garment, comprising:
i) at least one collector located close enough to the garment to catch odors emanating from the garment, comprising at least one flap having a plurality of holes for air flow;
ii) a piping system connected to the collector, wherein the piping system comprises piping surrounding a plurality of silica pellets; and
iii) a fan at an end of the piping system opposite the collector, such that the fan pulls air from around the garment into the holes in the collector, wherein the silica pellets absorb odors from the air.

25. The apparatus of claim 24, wherein the odor-absorbing system is removably attached to a wheelchair.

26. The apparatus of claim 24, wherein the collector is located on at least one arm of the wheelchair.

27. A method of collection and disposal of waste for a garment wearer, comprising the steps of:
a) providing a garment comprising a wearable, non-absorptive body having a lower, semi-rigid, waste collecting chamber for collecting feces, urine, and water away from a wearer during use, at least one inlet to receive water, and at least one outlet to dispose of waste;
b) connecting the inlet of the garment to a water supply line which distributes water to the garment, wherein an end of the water supply line opposite the inlet of the garment is connected to a portable cleaning unit;
c) connecting the outlet to a waste supply line which takes water and waste away from the garment, wherein an end of the waste supply line opposite the outlet of the garment is connected to the portable cleaning unit; and
d) collecting waste and dirty water in the portable cleaning unit through the waste supply line.

28. The method of claim 27, further providing, before step (d), the step of providing clean water to the garment from the water supply line.

29. A method of collection and disposal of waste for a garment wearer, wherein the garment comprises a wearable, non-absorptive body having a lower, semi-rigid, waste collecting chamber for collecting feces, urine, and water away from a wearer during use, at least one inlet to receive water, and at least one outlet to dispose of waste, comprising the steps of:
a) removing the garment from the wearer;
b) placing the garment on a ball-shaped form which allows the garment to retain its shape, wherein the ball-shaped form has at least one water supply line to provide water and at least one waste supply line to flush out the garment, wherein the ball-shaped form is located in a housing unit; and
c) cleaning the garment by the substeps of comprises the substeps of:
i) dispersing water into the inlet of the garment through the water supply line in the ball-shaped form;
ii) allowing water and waste to fall into the waste-collecting chamber; and
iii) moving water and waste from the waste-collecting chamber out of the garment through the outlet using the waste supply line in the ball-shaped form.

30. The method of claim 29, further comprising, after step (c), the step of sterilizing the garment.

31. The method of claim 29, further comprising, after step (c), the step of air drying the garment.

32. The method of claim 29, wherein substeps i) and iii) are accomplished by plumbing connected to the housing unit.

33. A system for controlling odors, comprising:
a) at least one collector located close enough to an odor-causing agent to catch odors emanating from the odor-causing agent, comprising a flap having a plurality of holes for air flow;
b) a piping system connected to the collector, wherein the piping system comprises piping surrounding a plurality of silica pellets; and
c) a fan at an end of the piping system opposite the collector, wherein the fan pulls air from the odor-causing agent through the piping system, wherein the silica pellets absorb odors from the air.

34. The apparatus of claim 33, wherein the odor-causing agent comprises a garment for disposal of human waste comprising a wearable, non-absorptive body having a lower, semi-rigid, waste collecting chamber for collecting feces, urine, and water away from a wearer during use, at least one inlet to receive water, and at least one outlet to dispose of waste

35. The apparatus of claim 33, wherein the odor-absorbing system is removably attached to a wheelchair.

36. The apparatus of claim 35, wherein the collector is located on an arm of the wheelchair.
